(19) United States
(12) Patent Application Publication
(10) Pub. No.: US 2007/0078215 A1
(43) Pub. Date: Apr. 5, 2007

(54) Dispersant for carbon nanotube and composition comprising the same

(76) Inventors: Seon Mi Yoon, Yongin-Si (KR); Eun Sung Lee, Seoul (KR); Jae Young Choi, Suwon-Si (KR); Do Yun Kim, Seongnam-Si (KR)

Correspondence Address: CANTOR COLBURN, LLP, 55 GRIFFIN ROAD SOUTH, BLOOMFIELD, CT, 06002, US

(21) Appl. No.: 11/352,137

(22) Filed: Feb. 10, 2006

(30) Foreign Application Priority Data
Oct. 5, 2005 (KR) 2005-93352

(51) Int. Cl. C08K 3/04; C07C 43/00

(52) U.S. Cl. 524/495; 106/472; 568/606

(57) ABSTRACT

A dispersant for a carbon nanotube and a composition comprising the same are provided, wherein the dispersant is comprised of a structure including a head part composed of an electron-rich atom and an aromatic ring having a high affinity for the carbon nanotube and a tail part having an affinity for a dispersion medium, and thus exhibits excellent stabilizing and dispersing effects of the carbon nanotube in a variety of dispersion media including organic solvents, water or mixtures thereof. Use of the dispersant in accordance with the present invention enables convenient preparation of carbon nanotube compositions necessary for a variety of industrial fields such as emitters of field emission displays (FEDs), carbon nanotube inks, printable carbon nanotubes and the like.

### BACKGROUND OF THE INVENTION

This non-provisional application claims priority under 35 U.S.C. §119(a) on Korean Patent Application No. 2005-93352 filed on Oct. 5, 2005, which is herein incorporated by reference.

1. Field of the Invention

The present invention relates to a dispersant for a carbon nanotube and a composition comprising the same. More specifically, the present invention relates to a dispersant having a structure including a head part composed of an electron-rich atom and an aromatic ring having a high affinity for the carbon nanotube and a tail part having affinity for a dispersion medium and thus having improved dispersibility of the carbon nanotube in various solvents, and a composition comprising the same.

2. Description of the Related Art

Carbon nanotubes (CNTs), materials in which carbon atoms are positioned in a hexagonal honeycomb-like pattern to create a tube form, are highly anisotrophic, exhibit various structural forms such as single-walled carbon nanotubes (SWNTs), multi-walled carbon nanotubes (MWNTs), bundles of carbon nanotubes, etc, and have a very small tube diameter in a nanometer (nm=10⁻⁹ m) range. In addition, carbon nanotubes (CNTs) have superior mechanical properties, electrical selectivity and excellent field emission properties and are high-efficiency hydrogen storage media. The carbon nanotubes can be either a semiconductor or a metal depending on how the tube is rolled, and energy gaps thereof vary depending upon diameter. In addition, carbon nanotubes have a quasi-one-dimensional structure and thus exert unique quantum effects. Methods known to synthesize carbon nanotubes include arc-discharge, thermal decomposition, laser vaporization, plasma enhanced chemical vapor deposition, thermal chemical vapor deposition, electrolysis and the like. In addition, carbon nanotubes also exhibit high electrical conductivity and thus are currently used to form conductive films, and a great deal of attention has been focused on their potential uses in the near future for field emission displays (FEDs) and probes for a scanning probe microscope (SPMs). Therefore, a great deal of intensive research is being actively undertaken as to the feasibility of such applications.

Meanwhile, as carbon nanotubes are generally obtained together with carbon particles such as carbon black during production thereof, it is necessary to separate and purify carbon particles from mixtures of carbon nanotubes and carbon particles. In addition, in order to use carbon nanotubes to form conductive films or prepare other devices, it may be necessary to precede preparation of a paste by mixing the carbon nanotubes with conventional solvents and binders. In order to purify the carbon nanotubes or prepare a paste thereof, it is necessary that the carbon nanotubes are dissolved in a suitable dispersion medium. Particularly, for dispersion of the carbon nanotubes involved in use and application thereof, selection of a dispersant to be used should be more carefully considered because a cohesive force between particles is very large from the viewpoint of the properties of the carbon nanotubes.

The dispersant is a surfactant and is composed of a head part and a tail part. The head part of the dispersant should have an affinity for a surface of a dispersoid which is a material to be dispersed, while the tail part thereof should have an affinity for a dispersion solvent, i.e., a dispersion medium. In addition, in order to be a good dispersant, it should serve as a barrier against collision between particles.

Examples of conventional dispersants for the carbon nanotubes include aqueous dispersants such as sodium dodecyl benzen sulfonate (NaDDBS), sodium dodecyl sulfonate, TX-100 and polyvinyl pyrrolidone. NaDDBS is known as the most superior dispersant. However, the above-mentioned aqueous dispersants all exhibit good dispersion of carbon nanotubes in water, but disadvantageously exhibit poor dispersion effects in organic solvents.

In addition, although there is yet no well-known organic dispersant, Korean Patent Publication Laid-open No. 2004-0039425 and Japanese Patent Publication Laid-open No. 2004-00339301 disclose a fact that carbon nanotubes can be readily dispersed in organic solvents using a conjugated polymer such as polythiophene-based polymer. However, these patents are contrived for providing organic semiconductor materials having high mobility of carriers, and thus are completely different from the present invention in terms of the object of the invention. In addition, the above-mentioned inventions employ the polythiophene-based polymer, a molecular weight of which is not controlled, and thus suffer from disadvantages in that the number of utilizable dispersion media is limited to 2 or 3 species and the intrinsic viscosity of the polymer having a high molecular weight inhibits dispersion of particles, thus leading to many limitations in performing processes.

As such, recently, there is a need for the development of a novel dispersant for carbon nanotubes, which is capable of easily dispersing carbon nanotubes in various solvents including organic solvents, aqueous solvents and mixtures thereof.

### SUMMARY OF THE INVENTION

Therefore, the present invention has been made in view of the above problems, and it is an object of the present invention to provide a dispersant for a carbon nanotube, comprising a structure including a head part composed of an electron-rich atom and an aromatic ring having a high affinity for the carbon nanotube and a tail part having an affinity for a dispersion medium, and thus having excellent stabilizing and dispersing effects of the carbon nanotube in various kinds of solvents.

It is another object of the present invention to provide a composition comprising the above-mentioned dispersant which is thus capable of improving dispersion of a carbon nanotube.

In accordance with an aspect of the present invention, the above and other objects can be accomplished by the provision of a dispersant for a carbon nanotube, comprising:

a head part selected from the group consisting of —SH, —NH₂ and a group represented by Formula 1 below:

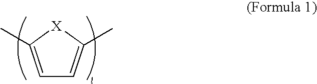

wherein X represents S, NH or O, and

l represents an integer from 1 to 60; and

a tail part represented by Formula 2 below:

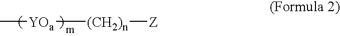

wherein Y is selected from the group consisting of substituted or unsubstituted C1-C10 alkylene, substituted or unsubstituted C1-C10 alkenylene, substituted or unsubstituted C1-C10 alkynylene and substituted or unsubstituted C6-C20 arylalkylene,

Z is selected from the group consisting of —H, —CH₃, —OH or a carboxylic acid or a salt thereof, a sulfonic acid or a salt thereof and phosphoric acid or a salt thereof,

a represents 0 or 1,

m represents an integer from 1 to 9, and

n represents an integer from 0 to 9.

In accordance with another aspect of the present invention, there is provided a composition comprising the above-mentioned dispersant, a carbon nanotube and a dispersion medium selected from an organic solvent, water and a mixture thereof.

The composition in accordance with the present invention may contain 0.001 to 10 parts by weight of a dispersant, 0.01 to 5 parts by weight of a carbon nanotube, and the balance of a dispersion medium selected from an organic solvent, water and a mixture thereof, based on 100 parts by weight of the composition.

A mixing weight ratio of the carbon nanotube dispersant in the composition is preferably in a range of 1:0.001 to 1:10.

In addition, the composition may further contain one or more additives selected from the group consisting of an organic binder, a photosensitive monomer, a photoinitiator, a viscosity-adjusting agent, a storage stabilizer, a wetting agent and an acid or base.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing measurement results of the absorbance at 800 nm of carbon nanotube solutions with respect to species of head parts of dispersants in accordance with the present invention;

FIG. 2 is a graph showing measurement results of the absorbance at 800 nm of carbon nanotube solutions with respect to species of tail parts of dispersants in accordance with the present invention;

FIG. 3 is graph showing measurement results of the absorbance at 800 nm of carbon nanotube solutions with respect to species of dispersion media and dispersants in accordance with the present invention;

FIG. 4 is a SEM of a surface of a carbon nanotube film prepared with a carbon nanotube paste composition which is obtained using a dispersant in accordance with the present invention;

FIG. 5 is a SEM of a surface of a carbon nanotube film prepared with a carbon nanotube paste composition without a dispersant in accordance with the present invention;

FIG. 6 is a photograph showing experimental results of solubility of a dispersant in accordance with the present invention (poly(3-hexylthiophene)) having a molecular weight of 87,000; and

FIG. 7 is a photograph showing experimental results of solubility of a dispersant in accordance with the present invention (poly(3-hexylthiophene)) having a molecular weight of 6,000.

### DESCRIPTION OF THE PREFERRED EMBODIMENTS

Hereinafter, a dispersant for a carbon nanotube in accordance with the present invention will be described in more detail.

The head part of the dispersant in accordance with the present invention is composed of an electron-rich atom, for example sulfur and nitrogen, such as —SH, —NH₂ and a group represented by Formula 1 below and an aromatic ring having a high affinity for carbon atoms of the carbon nanotube. Therefore, the head part of the dispersant can easily provide electrons to the carbon nanotubes, can form a π-π coupling with the carbon nanotubes, and can be adsorbed on carbon nanotube particles in a comb structure wrapping fashion, thereby making it possible to easily disperse carbon nanotubes in any dispersion medium.

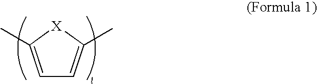

wherein X represents S, NH or O, and

l represents an integer from 1 to 60;

The tail part of the dispersant, which is bound to the head part, is composed of a structure of Formula 2 below having a high affinity for both an organic solvent and an aqueous solvent. Therefore, the dispersant including such a tail part in accordance with the present invention allows for carbon nanotubes to be easily dispersed in a wide range of various dispersion media including an organic solvent, water, a mixture of two or more organic solvents and a mixture of one or more polar solvents and water.

The tail part spreads in all directions from around the head part and thereby imparts steric hindrance and electrostatic repulsion, thus serving to prevent collision and aggregation between carbon nanotube particles.

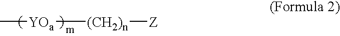

wherein Y is selected from the group consisting of substituted or unsubstituted C1-C10 alkylene, substituted or unsubstituted C1-C10 alkenylene, substituted or unsubstituted C1-C10 alkynylene and substituted or unsubstituted C6-C20 arylalkylene,

Z is selected from the group consisting of —H, —CH₃, —OH or a carboxylic acid or a salt thereof, a sulfonic acid or a salt thereof and phosphoric acid or a salt thereof,

a represents 0 or 1,

m represents an integer from 1 to 9, and

n represents an integer from 0 to 9.

In Formula 2, when a is 0, increased hydrophobicity leads to good dispersion of carbon nanotubes primarily in organic solvents. In contrast, when a is 1, increased hydrophilicity leads to good dispersion of carbon nanotubes primarily in polar solvents, water or mixtures thereof. Such dispersion effects result from steric hindrance effects.

Furthermore, introduction of a carboxylic acid or a salt thereof, a sulfonic acid or a salt thereof, or phosphoric acid or a salt thereof, each capable of being charged, into Z may induce electrostatic repulsion and therefore it is possible to more effectively disperse carbon nanotubes in polar solvents, water or mixtures thereof.

In Formula 2, specific examples of unsubstituted C1-C10 alkylene include methylene, ethylene, propylene, isobutylene, sec-butylene, pentylene, iso-amylene and hexylene. In alkylene group, one or more hydrogen atoms may be substituted with a halogen atom, hydroxy, nitro, cyano, amino, amidino, hydrazine, hydrazone, a carboxylic acid or a salt thereof, a sulfonic acid or a salt thereof and phosphoric acid or a salt thereof.

As used herein, the term “unsubstituted C1-C10 alkenylene or alkynylene” refers to a structure that contains a carbon-carbon double bond or triple bond at the middle or ends of alkylene as defined above. Specifically, examples of alkenylene or alkynylene include ethylene, propylene, butylenes, hexylene and acetylene, wherein one or more hydrogen atoms thereof may be substituted with a halogen atom, hydroxy, nitro, cyano, amino, amidino, hydrazine, hydrazone, a carboxylic acid or a salt thereof, a sulfonic acid or a salt thereof and phosphoric acid or a salt thereof.

As used herein, the term “arylalkylene” refers to a structure in which a portion of hydrogen atoms, from arylene which is a C6-C20 carbocyclic aromatic system including one or more rings, is substituted with a radical such as lower alkylene, for example methylene, ethylene or propylene. For example, benzylene and phenylethylene may be mentioned. Similarly, in arylalkylene group, one or more hydrogen atoms may be substituted with a halogen atom, hydroxy, nitro, cyano, amino, amidino, hydrazine, hydrazone, a carboxylic acid or a salt thereof, a sulfonic acid or a salt thereof and phosphoric acid or a salt thereof.

The preferred examples of the tail part that can be used in the present invention include, but are not limited to, C3-C20 polyethylene oxide and C4-C20 polypropylene oxide.

The preferred examples of dispersants having the above-mentioned structure in accordance with the present invention may include, but are not limited to, poly(3-hexylthiophene) (having a molecular weight of less than 10,000), 3-hexylthiophene, 3-dodecylthiophene, poly(3-pentadecylpyrrole), hexylpyrrole, dodecylpyrrole, hexylthiol, dodecanethiol, polyhexylaniline,

a compound represented by Formula 3 below:

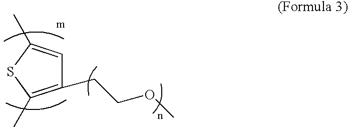

wherein m represents an integer from 1 to 60, and n represents an integer from 1 to 12; and

a compound represented by Formula 4 below:

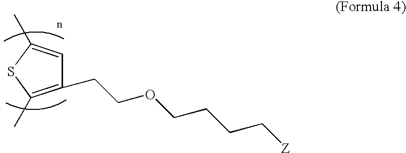

wherein Z is selected from the group consisting of —H, —CH₃, —OH or a carboxylic acid or a salt thereof, a sulfonic acid or a salt thereof and phosphoric acid or a salt thereof, and

n represents an integer from 1 to 60.

Preferably, the dispersant having such a structure composed of the head part and tail part in accordance with the present invention has a molecular weight of less than 10,000. If the dispersant has a lower molecular weight less than 10,000, solubility thereof is increased and therefore species of utilizable dispersion media are further extended and viscosity of the dispersant itself is also lowered, thus being more suitable for preparation processes.

Hereinafter, a composition containing the dispersant in accordance with the present invention will be reviewed.

The composition in accordance with the present invention comprises the dispersant in accordance with the present invention; a carbon nanotube; and a dispersion medium selected from an organic solvent, water and a mixture thereof.

The composition in accordance with the present invention may contain 0.001 to 10 parts by weight of a dispersant, 0.01 to 5 parts by weight of a carbon nanotube, and the balance of a dispersion medium selected from an organic solvent, water and a mixture thereof, based on 100 parts by weight of the composition.

Herein, the mixing weight ratio of the carbon nanotube dispersant is preferably in a range of 1:0.001 to 1:10. This is because where the amount of the dispersant is smaller than the above mixing weight ratio range, it is impossible to achieve suitable dispersion effects of the carbon nanotube, and in contrast, where the amount of the dispersant is greater than the above range, this may cause negative effects due to the viscosity of the dispersant itself.

The carbon nanotube that is utilizable in the present invention can be selected from the group consisting of single-walled carbon nanotubes (SWNTs), double-walled carbon nanotubes (DWNTs), multi-walled carbon nanotubes (MWNTs), bundles of carbon nanotubes, and any combination thereof.

The dispersion media that is utilizable in the present invention includes, but is not limited to, for example an organic solvent, water, a mixture of two or more organic solvents and a mixture of one or more polar solvents and water.

Examples of the organic solvent include alcohols such as methyl alcohol, ethyl alcohol, n-propyl alcohol, isopropyl alcohol, n-butyl alcohol, sec-butyl alcohol, t-butyl alcohol, isobutyl alcohol and diacetone alcohol; ketones such as acetone, methyl ethyl ketone and methyl isobutyl ketone; glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; glycol ethers such as ethylene glycol monomethyl ether and triethylene glycol monoethyl ether; glycol ether acetates such as propylene glycol monomethyl ether acetate (PGMEA); acetates such as ethyl acetate, butoxyethoxy ethyl acetate, butyl carbitol acetate (BCA) and dihydroterpineol acetate (DHTA); terpineols; trimethyl pentanediol monoisobutyrate (TEXANOL); dichloroethene (DCE); and 1-methyl pyrrolidone (NMP). These organic solvents may be used alone or in any combination.

Meanwhile, if necessary, the composition in accordance with the present invention may further contain one or more additives selected from the group consisting of an organic binder, a photosensitive monomer, a photoinitiator, a viscosity-adjusting agent, a storage stabilizer, a wetting agent and an acid or base, within a range that they do not damage physical properties of the composition.

The content of the additive may be in a range of 0.1 to 60 parts by weight, based on 100 parts by weight of the composition in accordance with the present invention.

The organic binder that is utilizable in the present invention includes, but is not limited to, for example cellulose including ethylcellulose, styrene, styrene-acrylate copolymer, polyvinylbutyral, polyvinyl alcohol and polypropylene carbonate. Preferably, cellulose-based binders such as ethylcellulose may be used alone or in any combination thereof.

As the photosensitive monomer and photoinitiator, those conventionally used in the art may be used without particular limitation. Specific examples of the photosensitive monomer may include thermally degradable acrylate monomers, benzophenone monomers, acetphenone monomers and thioxanthone monomers.

As the viscosity-adjusting agent and storage stabilizer, those conventionally used in the art may also be used without particular limitation. Specific examples of the viscosity-adjusting agent may include casein and carboxymethylcellulose.

Similarly, as the wetting agent, those conventionally used in the art may also be used without particular limitation. Specific examples of the wetting agent may include polyhydric alcohols such as glycerin, ethylene glycol, diethylene glycol, triethylene glycol, propylene glycol, dipropylene glycol, 1,3-butanediol, 1,4-butanediol, 1,5-pentanediol, 1,2-hexanediol and 2-methyl-2-pentanediol.

The composition in accordance with the present invention may further contain an acid or base, as discussed hereinbefore. Such an acid or base increases the solubility of the dispersant in water and polar solvents and imparts electrostatic repulsion force to the dispersed carbon nanotube particles, thereby stabilizing the dispersed state of carbon nanotubes. Herein, the acid that can be used in the present invention may include, for example hydrochloric acid, sulfuric acid, nitric acid, acetic acid and carbonic acid. Examples of the base utilizable in the present invention may include sodium hydroxide, potassium hydroxide, calcium hydroxide and ammonium hydroxide.

The composition of the present invention as constituted above can be applied to a variety of industrial fields which can use aqueous or oily carbon nanotube compositions. Specifically, the composition of the present invention can be used for preparation of emitters of field emission displays (FEDs), carbon nanotube inks, printable carbon nanotubes and the like.

### EXAMPLES

Now, the present invention will be described in more detail with reference to the following examples. These examples are provided only for illustrating the present invention and should not be construed as limiting the scope and spirit of the present invention.

Determination of Dispersion Effects of Carbon Nanotube with Respect to Species of Head Parts of Dispersants in Accordance with the Present Invention

### Example 1

20 mg of poly(3-hexylthiophene) as a dispersant was dissolved in 20 ml of chloroform, and 2 mg of multi-walled carbon nanotubes (MWNTs) was added to the resulting solution which was then dispersed in a sonicbath for 10 hours and centrifuged at 5600 rpm for 5 min, thereby preparing a carbon nanotube solution.

### Example 2

A carbon nanotube solution was prepared in the same manner as in Example 1, except that 3-hexylthiophene was used as a dispersant.

### Example 3

A carbon nanotube solution was prepared in the same manner as in Example 1, except that 3-dodecylthiophene was used as a dispersant.

### Example 4

A carbon nanotube solution was prepared in the same manner as in Example 1, except that 3-dodecanethiol was used as a dispersant.

### Comparative Example 1

A carbon nanotube solution was prepared in the same manner as in Example 1, except that a dispersant was not used.

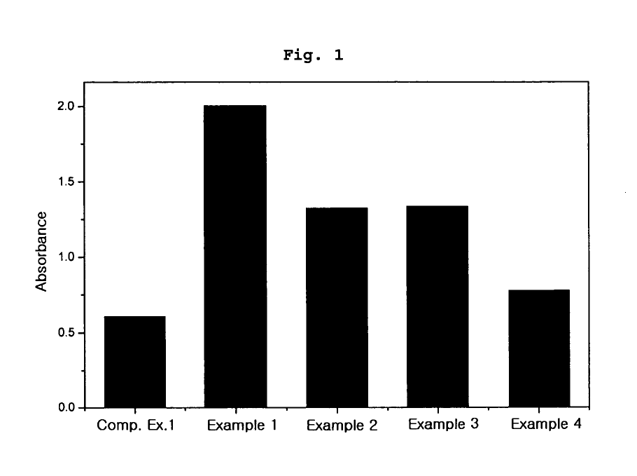

Respective carbon nanotube solutions prepared in Examples 1 through 4 and Comparative Example 1 were centrifuged to remove aggregated powder and the absorbance thereof was measured using a UV-Vis-spectroscopy (JASCO V-560, Absorbance mode, Scanning speed: 400 nm/min) at 800 nm. The results thus obtained are shown in FIG. 1. Herein, a dispersant solution containing no carbon nanotube was used as a standard solution.

Referring to FIG. 1, Examples 1 through 4 using the dispersants in accordance with the present invention exhibited a higher absorbance as compared to Comparative Example 1 without using the same, and thus it can be confirmed that the dispersants of the present invention disperse carbon nanotubes well in organic solvents and those having thiophene or polythiophene as the head part (Examples 1 through 3) among the dispersants exhibit superior dispersion effects.

Determination of Dispersion Effects of Carbon Nanotube with Respect to Species of Tail Parts of Dispersants in Accordance with the Present Invention

### Example 5

A carbon nanotube solution was prepared using a compound represented by Formula 5 below and composed of a polythiophene head part and a polyethylene oxide tail part as a dispersant. Specifically, 20 mg of the above dispersant was dissolved in 20 ml of terpineol, and 2 mg of single-walled carbon nanotubes (SWNTS) was added to the resulting solution which was then dispersed in a sonicbath for 10 hours and centrifuged at 5600 rpm for 5 min, thereby obtaining a carbon nanotube solution.

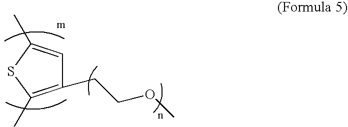

wherein m is 50 and n is 9.

### Example 6

A carbon nanotube solution was prepared in the same manner as in Example 5, except that poly(3-hexylthiophene) (a molecular weight of 6,000) having the same polythiophene head part as the dispersant of Example 5 and having alkyl(hexyl) tail as a tail part was used as a dispersant.

### Comparative Example 2

A carbon nanotube solution was prepared in the same manner as in Example 5, except that a dispersant was not used.

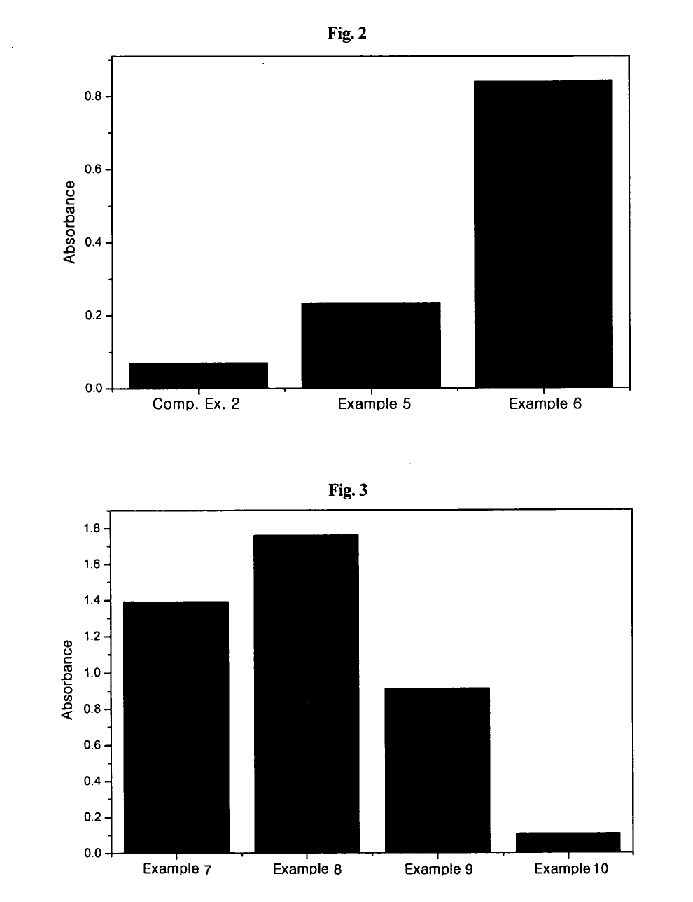

Respective carbon nanotube solutions prepared in Examples 5 and 6 and Comparative Example 2 were centrifuged to remove aggregated powder and absorbance thereof was measured using a UV-Vis-spectroscopy (JASCO V-560, Absorbance mode, Scanning speed: 400 nm/min) at 800 nm. The results thus obtained are shown in FIG. 2.

Referring to FIG. 2, both of Example 5 using the dispersant having the polyethylene oxide tail part and Example 6 using the dispersant having the alkyl tail part exhibited higher absorbance as compared to Comparative Example 2 without dispersants, and thus it can be confirmed that the dispersants of the present invention disperse carbon nanotubes well in organic solvents, regardless of tail part species. Here, the reason why the dispersant having the alkyl tail part among the dispersants in accordance with the present invention exhibits higher absorbance is that the dispersant having the polyethylene oxide tail part has lower solubility in a terpineol solvent than that of the dispersant having the alkyl tail part.

Determination of Dispersion Effects of Carbon Nanotube with Respect to Species of Dispersion Media and Dispersants in Accordance with the Present Invention

### Example 7

A carbon nanotube solution was prepared in the same manner as in Example 5, except that a compound represented by Formula 6 below was used as a dispersant and water was used as a dispersion medium.

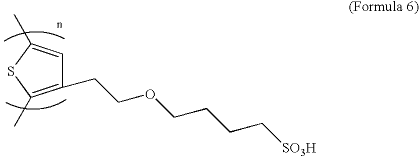

wherein n is 50.

### Example 8

A carbon nanotube solution was prepared in the same manner as in Example 7, except that a mixture of water (4 ml) and ethyl alcohol (16 ml) was used as a dispersion medium.

### Example 9

A carbon nanotube solution was prepared in the same manner as in Example 7, except that poly(3-pentadecylpyrrole) was used as a dispersant.

### Example 10

A carbon nanotube solution was prepared in the same manner as in Example 7, except that polyhexylaniline was used as a dispersant.

Respective carbon nanotube solutions prepared in Examples 7 through 10 were centrifuged to remove aggregated powder and the absorbance thereof was measured using a UV-Vis-spectroscopy (JASCO V-560, Absorbance mode, Scanning speed: 400 nm/min) at 800 nm. The results thus obtained are shown in FIG. 3.

Referring to FIG. 3, when various dispersants in accordance with the present invention were used in water or a mixture of water and a polar solvent, all cases have exhibited higher absorbance and thus it can be quantitatively confirmed that a range of dispersion media to which the dispersants of the present invention can be applied is various including organic solvents as well as water and mixtures of polar solvents and water.

Determination of Changes in Viscosity of Carbon Nanotube Paste Composition with or without Use of Dispersant in Accordance with the Present Invention, and SEM Thereof

### Example 11

8.335 g of ethylcellulose as an organic binder was dissolved in 13.775 g of a terpineol solvent to prepare a binder solution. 0.019 g of a dispersant of Example 5 and 0.38 g of multi-walled carbon nanotubes (MWNTs) were added to the resulting binder solution which was then mixed using a ball mill for 10 hours, thereby preparing a carbon nanotube paste composition.

### Comparative Example 3

A carbon nanotube paste composition was prepared in the same manner as in Example 11, except that a dispersant was not used.

(1) Measurement of Viscosity

Changes in viscosity of the respective carbon nanotube paste compositions prepared in Example 11 and Comparative Example 3 were measured with increasing shear rates. In this connection, the viscosity of the composition at different shear rates was measured on a viscometer (Brookfield RV II, Brookfield Engineering Laboratories, Inc., Stoughton, Mass., USA) (at different shear rates) using a spindle no. 14 at a temperature of 24.5 to 25.5° C. for 30 sec.

As a result, the composition of Comparative Example 3 exhibited a viscosity of 66000 cps at 2 rpm and 23400 cps at 20 rpm, respectively, while the composition of Example 11 using the dispersant in accordance with the present invention exhibited a lower viscosity of 46500 cps at 2 rpm and 14325 cps at 20 rpm, respectively, thus confirming that the composition using the dispersant in accordance with the present invention exhibits pronounced viscosity-reducing effects as compared to the composition of Comparative Example.

(2) Preparation of Carbon Nanotube Film and SEM Thereof

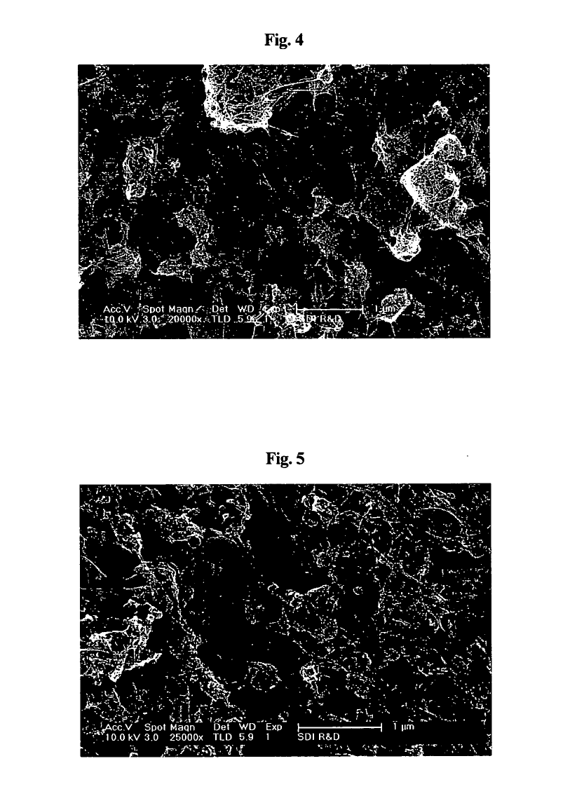

The carbon nanotube paste compositions prepared in Example 11 and Comparative Example 3 were printed to a thickness of 30 μm on glass substrates, respectively, and were fired at 380° C. in the air to thereby obtain carbon nanotube films. Surfaces of the thus-obtained carbon nanotube films were photographed under a scanning electron microscope. The results thus obtained are shown in FIGS. 4 and 5.

Referring to FIGS. 4 and 5, it can be confirmed that the carbon nanotube film (see FIG. 4), which was prepared with the carbon nanotube paste composition obtained using the dispersant in accordance with the present invention, exhibits homogeneous and good dispersion of carbon nanotubes, as compared to the carbon nanotube film (see FIG. 5) which was prepared with the carbon nanotube paste composition of Comparative Example 3 using no dispersant.

Experiment of Solubility of Dispersant in Accordance with the Present Invention with Respect to Molecular Weight

Kinds of solvents to which a dispersant can be applied are limited depending upon a molecular weight of the dispersant. This is because desired effects of the dispersant cannot be obtained unless the dispersant itself is dissolved in the solvent to be used. Therefore, as will be described hereinafter, the molecular weight of the dispersant of the present invention was adjusted and then the solubility thereof was measured in the corresponding various solvents.

### Experimental Examples 1 through 13

2 mg of poly(3-hexylthiophene) having a molecular weight of 87,000 as a dispersant was introduced into 20 ml of each solvent listed in Table 1 below, and the resulting mixture was homogenized in an ultrasonic homogenizer for about 4 hours, thereby obtaining 13 dispersant solutions.

### Experimental Examples 14 through 25

2 mg of poly(3-hexylthiophene) having a molecular weight of 6000 as a dispersant was introduced into 20 ml of each solvent listed in Table 1 below, and the resulting mixture was homogenized in an ultrasonic homogenizer for about 15 min, thereby obtaining 12 dispersant solutions.

TABLE 1

| Experimental Ex. | Solvents |
|---|---|
| 1, 14 | Hexane |
| 2, 15 | Toluene |
| 3, 16 | Chloroform |
| 4, 17 | Dichloroethene (DCE) |
| 5, 18 | Tetrahydrofuran |
| 6, 19 | Methylisobutylketone |
| 7, 20 | Methyl alcohol |
| 8, 21 | Isopropyl alcohol |
| 9 | Propylene glycol monomethyl ether acetate (PGMEA) |
| 10, 22 | 1-methyl pyrrolidone (NMP) |
| 11, 23 | Butyl carbitol acetate (BCA) |
| 12, 24 | Terpineol |
| 13, 25 | Water |

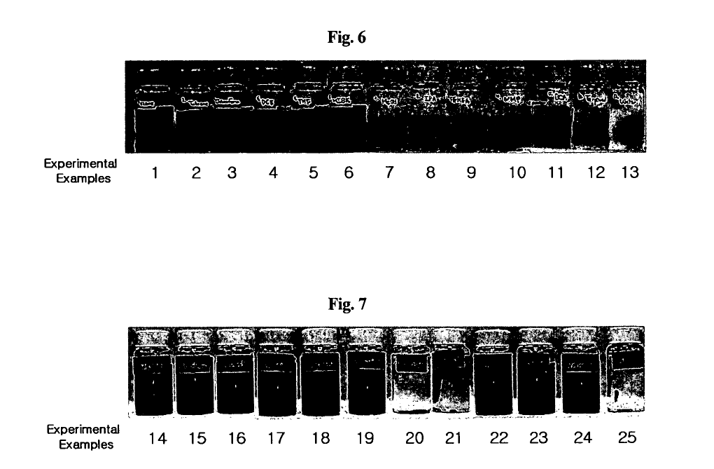

Degrees of dispersion in respective dispersant solutions the prepared in Experimental Examples 1 through 13 and the Experimental Examples 14 through 25 were photographed. The results thus obtained are shown in FIGS. 6 and 7. Referring to FIGS. 6 and 7, it can be seen that the dispersants having a molecular weight of 87000 were not dissolved easily even when the dissolution time was prolonged (FIG. 6), while the dispersants having a molecular weight of 6000 were dissolved well in most solvents within a very short period of dissolution time (FIG. 7). Therefore, it can be seen from the above results that the dispersants having a lower molecular weight, i.e., less than 10000, have a relatively high solubility as compared to the dispersants having a higher molecular weight, and thus kinds of utilizable solvents are various ranging from organic solvents to polar solvents.

As apparent from the above description, the dispersant provided by the present invention is comprised of a structure including a head part which is composed of an electron-rich atom and an aromatic ring having a high affinity for the carbon nanotube, thus capable of forming π-π coupling with the carbon nanotubes and capable of being adsorbed on carbon nanotube particles in a wrapping fashion; and a tail part having an affinity for an organic solvent and an aqueous solvent, and thus exhibits excellent stabilizing and dispersing effects of the carbon nanotube in a variety of dispersion media including organic solvents, water or mixtures thereof. Therefore, use of the dispersant in accordance with the present invention enables convenient preparation of carbon nanotube compositions necessary for a variety of industrial fields such as emitters of field emission displays (FEDs), carbon nanotube inks, printable carbon nanotubes and the like.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A dispersant for a carbon nanotube, comprising:
a head part selected from the group consisting of —SH, —NH₂ and a group represented by Formula 1 below: 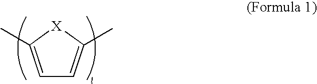
wherein X represents S, NH or O, and
I represents an integer from 1 to 60; and
a tail part represented by Formula 2 below: 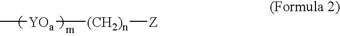
wherein Y is selected from the group consisting of substituted or unsubstituted C1-C10 alkylene, substituted or unsubstituted C1-C10 alkenylene, substituted or unsubstituted C1-C10 alkynylene and substituted or unsubstituted C6-C20 arylalkylene,
Z is selected from the group consisting of —H, —CH₃, —OH or a carboxylic acid or a salt thereof, a sulfonic acid or a salt thereof and phosphoric acid or a salt thereof,
a represents 0 or 1,
m represents an integer from 1 to 9, and
n represents an integer from 0 to 9.

2. The dispersant according to claim 1, wherein the dispersant is selected from the group consisting of poly(3-hexylthiophene) (having a molecular weight of less than 10,000), 3-hexylthiophene, 3-dodecylthiophene, poly(3-pentadecylpyrrole), hexylpyrrole, dodecylpyrrole, hexylthiol, dodecanethiol, polyhexylaniline,
a compound represented by Formula 3 below: 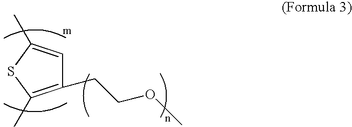
wherein m represents an integer from 1 to 60, and n represents an integer from 1 to 12; and
a compound represented by Formula 4 below: 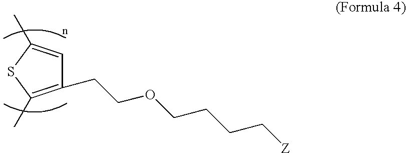
wherein Z is selected from the group consisting of —H, —CH₃, —OH or a carboxylic acid or a salt thereof, a sulfonic acid or a salt thereof and phosphoric acid or a salt thereof, and
n represents an integer from 1 to 60.

3. The dispersant according to claim 1, wherein the dispersant has a molecular weight of less than 10,000.

4. The dispersant according to claim 1, wherein the tail part is C3-C20 polyethylene oxide or C4-C20 polypropylene oxide.

5. A composition comprising the dispersant of claim 1, a carbon nanotube, and a dispersion medium selected from an organic solvent, water and a mixture thereof.

6. The composition according to claim 5, wherein the composition contains 0.001 to 10 parts by weight of a dispersant, 0.01 to 5 parts by weight of a carbon nanotube, and the balance of a dispersion medium selected from an organic solvent, water and a mixture thereof, based on 100 parts by weight of the composition.

7. The composition according to claim 5, wherein the mixing weight ratio of the carbon nanotube : dispersant is in the range of 1:0.001 to 1:10.

8. The composition according to claim 5, wherein the carbon nanotube is selected from the group consisting of a single-walled carbon nanotube (SWNT), a double-walled carbon nanotube (DWNT), a multi-walled carbon nanotube (MWNT), a bundle of carbon nanotube and any combination thereof.

9. The composition according to claim 5, wherein the organic solvent is selected from the group consisting of alcohols such as methyl alcohol, ethyl alcohol, n-propyl alcohol, isopropyl alcohol, n-butyl alcohol, sec-butyl alcohol, t-butyl alcohol, isobutyl alcohol and diacetone alcohol; ketones such as acetone, methyl ethyl ketone and methyl isobutyl ketone; glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; glycol ethers such as ethylene glycol monomethyl ether and triethylene glycol monoethyl ether; glycol ether acetates such as propylene glycol monomethyl ether acetate (PGMEA); acetates such as ethyl acetate, butoxyethoxy ethyl acetate, butyl carbitol acetate (BCA) and dihydroterpineol acetate (DHTA); terpineols; trimethyl pentanediol monoisobutyrate (TEXANOL); dichloroethene (DCE); 1-methyl pyrrolidone (NMP) and any combination thereof.

10. The composition according to claim 5, wherein the composition further contains one or more additives selected from the group consisting of an organic binder, a photosensitive monomer, a photoinitiator, a viscosity-adjusting agent, a storage stabilizer, a wetting agent and an acid or base.

11. The composition according to claim 10, wherein the content of the additive is in the range of 0.1 to 60 parts by weight, based on 100 parts by weight of the composition.

12. The composition according to claim 10, wherein the organic binder is selected from the group consisting of cellulose including ethylcellulose, styrene, styrene-acrylate copolymer, polyvinylbutyral, polyvinyl alcohol, polypropylene carbonate and any combination thereof.

13. A composition comprising the dispersant of claim 2, a carbon nanotube, and a dispersion medium selected from an organic solvent, water and a mixture thereof.

14. A composition comprising the dispersant of claim 3, a carbon nanotube, and a dispersion medium selected from an organic solvent, water and a mixture thereof.

15. A composition comprising the dispersant of claim 4, a carbon nanotube, and a dispersion medium selected from an organic solvent, water and a mixture thereof.

16. The composition according to claim 13, wherein the composition contains 0.001 to 10 parts by weight of a dispersant, 0.01 to 5 parts by weight of a carbon nanotube, and the balance of a dispersion medium selected from an organic solvent, water and a mixture thereof, based on 100 parts by weight of the composition.

17. The composition according to claim 14, wherein the composition contains 0.001 to 10 parts by weight of a dispersant, 0.01 to 5 parts by weight of a carbon nanotube, and the balance of a dispersion medium selected from an organic solvent, water and a mixture thereof, based on 100 parts by weight of the composition.

18. The composition according to claim 15, wherein the composition contains 0.001 to 10 parts by weight of a dispersant, 0.01 to 5 parts by weight of a carbon nanotube, and the balance of a dispersion medium selected from an organic solvent, water and a mixture thereof, based on 100 parts by weight of the composition.

19. The composition according to claim 13, wherein the mixing weight ratio of the carbon nanotube:dispersant is in the range of 1:0.001 to 1:10.

20. The composition according to claim 14, wherein the mixing weight ratio of the carbon nanotube:dispersant is in the range of 1:0.001 to 1:10.

21. The composition according to claim 15, wherein the mixing weight ratio of the carbon nanotube:dispersant is in the range of 1:0.001 to 1:10.

22. The composition according to claim 13, wherein the carbon nanotube is selected from the group consisting of a single-walled carbon nanotube (SWNT), a double-walled carbon nanotube (DWNT), a multi-walled carbon nanotube (MWNT), a bundle of carbon nanotube and any combination thereof.

23. The composition according to claim 14, wherein the carbon nanotube is selected from the group consisting of a single-walled carbon nanotube (SWNT), a double-walled carbon nanotube (DWNT), a multi-walled carbon nanotube (MWNT), a bundle of carbon nanotube and any combination thereof.

24. The composition according to claim 15, wherein the carbon nanotube is selected from the group consisting of a single-walled carbon nanotube (SWNT), a double-walled carbon nanotube (DWNT), a multi-walled carbon nanotube (MWNT), a bundle of carbon nanotube and any combination thereof.

25. The composition according to claim 13, wherein the organic solvent is selected from the group consisting of alcohols such as methyl alcohol, ethyl alcohol, n-propyl alcohol, isopropyl alcohol, n-butyl alcohol, sec-butyl alcohol, t-butyl alcohol, isobutyl alcohol and diacetone alcohol; ketones such as acetone, methyl ethyl ketone and methyl isobutyl ketone; glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; glycol ethers such as ethylene glycol monomethyl ether and triethylene glycol monoethyl ether; glycol ether acetates such as propylene glycol monomethyl ether acetate (PGMEA); acetates such as ethyl acetate, butoxyethoxy ethyl acetate, butyl carbitol acetate (BCA) and dihydroterpineol acetate (DHTA); terpineols; trimethyl pentanediol monoisobutyrate (TEXANOL); dichloroethene (DCE); 1-methyl pyrrolidone (NMP) and any combination thereof.

26. The composition according to claim 14, wherein the organic solvent is selected from the group consisting of alcohols such as methyl alcohol, ethyl alcohol, n-propyl alcohol, isopropyl alcohol, n-butyl alcohol, sec-butyl alcohol, t-butyl alcohol, isobutyl alcohol and diacetone alcohol; ketones such as acetone, methyl ethyl ketone and methyl isobutyl ketone; glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; glycol ethers such as ethylene glycol monomethyl ether and triethylene glycol monoethyl ether; glycol ether acetates such as propylene glycol monomethyl ether acetate (PGMEA); acetates such as ethyl acetate, butoxyethoxy ethyl acetate, butyl carbitol acetate (BCA) and dihydroterpineol acetate (DHTA); terpineols; trimethyl pentanediol monoisobutyrate (TEXANOL); dichloroethene (DCE); 1-methyl pyrrolidone (NMP) and any combination thereof.

27. The composition according to claim 15, wherein the organic solvent is selected from the group consisting of alcohols such as methyl alcohol, ethyl alcohol, n-propyl alcohol, isopropyl alcohol, n-butyl alcohol, sec-butyl alcohol, t-butyl alcohol, isobutyl alcohol and diacetone alcohol; ketones such as acetone, methyl ethyl ketone and methyl isobutyl ketone; glycols such as ethylene glycol, diethylene glycol, triethylene glycol, propylene glycol, butylene glycol, hexylene glycol, 1,3-propanediol, 1,4-butanediol, 1,2,4-butanetriol, 1,5-pentanediol, 1,2-hexanediol and 1,6-hexanediol; glycol ethers such as ethylene glycol monomethyl ether and triethylene glycol monoethyl ether; glycol ether acetates such as propylene glycol monomethyl ether acetate (PGMEA); acetates such as ethyl acetate, butoxyethoxy ethyl acetate, butyl carbitol acetate (BCA) and dihydroterpineol acetate (DHTA); terpineols; trimethyl pentanediol monoisobutyrate (TEXANOL); dichloroethene (DCE); 1-methyl pyrrolidone (NMP) and any combination thereof.

28. The composition according to claim 13, wherein the composition further contains one or more additives selected from the group consisting of an organic binder, a photosensitive monomer, a photoinitiator, a viscosity-adjusting agent, a storage stabilizer, a wetting agent and an acid or base.

29. The composition according to claim 14, wherein the composition further contains one or more additives selected from the group consisting of an organic binder, a photosensitive monomer, a photoinitiator, a viscosity-adjusting agent, a storage stabilizer, a wetting agent and an acid or base.

30. The composition according to claim 15, wherein the composition further contains one or more additives selected from the group consisting of an organic binder, a photosensitive monomer, a photoinitiator, a viscosity-adjusting agent, a storage stabilizer, a wetting agent and an acid or base.

31. The composition according to claim 28, wherein the content of the additive is in the range of 0.1 to 60 parts by weight, based on 100 parts by weight of the composition.

32. The composition according to claim 29, wherein the content of the additive is in the range of 0.1 to 60 parts by weight, based on 100 parts by weight of the composition.

33. The composition according to claim 30, wherein the content of the additive is in the range of 0.1 to 60 parts by weight, based on 100 parts by weight of the composition.

34. The composition according to claim 28, wherein the organic binder is selected from the group consisting of cellulose including ethylcellulose, styrene, styrene-acrylate copolymer, polyvinylbutyral, polyvinyl alcohol, polypropylene carbonate and any combination thereof.

35. The composition according to claim 29, wherein the organic binder is selected from the group consisting of cellulose including ethylcellulose, styrene, styrene-acrylate copolymer, polyvinylbutyral, polyvinyl alcohol, polypropylene carbonate and any combination thereof.

36. The composition according to claim 30, wherein the organic binder is selected from the group consisting of cellulose including ethylcellulose, styrene, styrene-acrylate copolymer, polyvinylbutyral, polyvinyl alcohol, polypropylene carbonate and any combination thereof.
